(19) United States
(12) Patent Application Publication
(10) Pub. No.: US 2018/0338937 A1
(43) Pub. Date: Nov. 29, 2018

(54) PHARMACEUTICAL PREPARATIONS

(71) Applicants: Analyticon Discovery GmbH, Potsdam (DE); B.R.A.I.N. Biotechnology Research and Information Network AG, Zwingenberg (DE)

(72) Inventors: Karsten SIEMS, Michendorf (DE); Katja RIEDEL, Bensheim (DE); Michael KROHN, Lorsch (DE)

(21) Appl. No.: 15/776,946

(22) PCT Filed: Dec. 11, 2016
(86) PCT No.: PCT/EP2016/080537

§ 371 Date: May 17, 2018

(30) Foreign Application Priority Data
Dec. 19, 2015 (EP) 15201444.5

(51) Int. Cl. A61K 31/19; A61P 29/00; A61K 31/616; A61K 31/167; A61K 31/404; A61K 31/381; A61K 9/00; A61K 47/12; A61K 47/42; A61K 47/26; A61K 47/18; A61K 47/38

(52) U.S. Cl. CPC A61K 31/19; A61P 29/00; A61K 31/616; A61K 31/167; A61K 31/404; A61K 47/38; A61K 9/0053; A61K 47/12; A61K 47/42; A61K 47/26; A61K 47/183; A61K 31/381

(57) ABSTRACT

A preparation is suggested, containing
(a) at least one NSAID active ingredient, and
(b) dehydroabietic acid or an extract containing dehydroabietic acid as a pharmaceutical product.

### FIELD OF THE INVENTION

The invention is in the field of pharmaceutics and relates to preparations with NSAID active ingredients and specific terpene derivatives, which are characterised by an improved oral administration and a better compatibility.

### STATE OF THE ART

The abbreviation NSAID (Non-Steroidal Anti-Inflammatory Drugs) summarises a group of non-opioid analgesics which are used for the systemic treatment of rheumatism due to their anti-inflammatory (antiphlogistic) effect. In contrast to this, glucocorticoids are referred to as steroidal anti-inflammatory drugs. The category was introduced in the late 1950s in order to mark the difference to ‘steroidal antirheumatic drugs’ with their, in part, severe side effects.

Nowadays, NSAID represent the most important group of analgesics, comprising such well-known substances as acetylsalicylic acid and ibuprofen. These substances do not only share the same mechanism of action, but also the circumstance that their oral uptake is made difficult by their bitter, partly astringent and metallic taste, which cannot be masked by sweeteners, or just a little, at best. In many cases, the adverse taste is even intensified by sweeteners such as, for example, acesulfame K or saccharin.

A further problem that is linked with the oral uptake of the NSAID active ingredients is their insufficient mucous membrane compatibility, which, when administered orally, leads to burning sensations and irritations in the oral cavity, specifically in the throat and on the tongue, producing a dry mouthfeel. Sometimes, these problems may have quite a long duration. In addition, damage to the gastric mucosa may occur.

The object of the present invention was therefore to remedy the disadvantageous features of the NSAID active ingredients, formulating them in a manner using suitable adjuvants such that liquid or solid pharmaceutical products are obtained, which are perfect in taste and have an improved mucous membrane compatibility at the same time.

### DESCRIPTION OF THE INVENTION

A first subject matter of the invention thus relates to a preparation, containing

(a) at least one NSAID active ingredient, and
(b) dehydroabietic acid or an extract containing dehydroabietic acid, as a pharmaceutical product.

A second subject matter is directed at a preparation, containing

(a) at least one NSAID active ingredient, and
(b) dehydroabietic acid or an extract containing dehydroabietic acid,
for use as a pharmaceutical product for the treatment and/or the prophylaxis of pain conditions, inflammatory conditions and rheumatic diseases.

Surprisingly, it was found that the formulation of NSAID active ingredients with dehydroabietic acid or extracts containing dehydroabietic acid (preferably in quantities of at least 20% by weight, more particularly at least 30% by weight and more particularly 40 to 60% by weight) fully meets the above complex requirements. Dehydroabietic acid is not only capable of masking the bitter, astringent and metallic taste of the NSAID, but can also noticeably improve the taste perception of formulations which contain NSAID along with sweeteners. In many cases, this entails a pleasant intensification of the aroma substances and the sweetness of the formulation.

A further important aspect is the fact that dehydroabietic acid also significantly improves the mucous membrane compatibility. During oral uptake, corresponding formulations do not cause any burning sensation at all, or very little, also the mouth moisture is not longer adversely affected. In total, patients perceive the uptake of a pharmaceutical product with NSAID-dehydroabietic acid significantly more pleasant, also with respect to the compatibility with the gastric mucosa.

NSAID Active Ingredients (Non-Steroidal Anti-Inflammatory Drugs)

The most important non-specific NSAID, which form group (a) of the invention, include the so-called COX-1/2 inhibitors, namely:

Group 1: Acetylsalicylic Acid Derivatives (1)

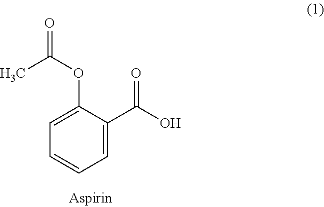

Group 2: Arylpropionic acid derivatives (2) to (6)

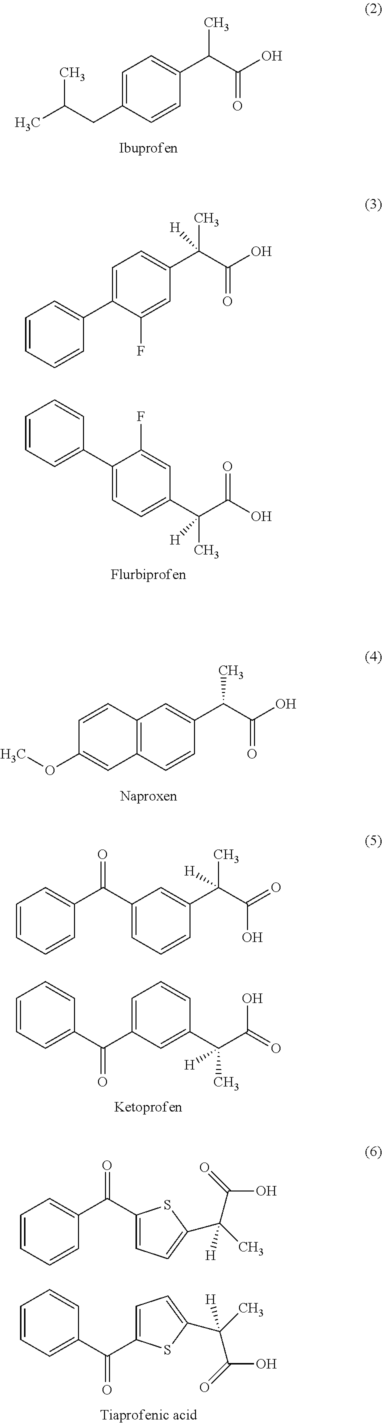

Group 3: Arylacetic Acid Derivatives (7)

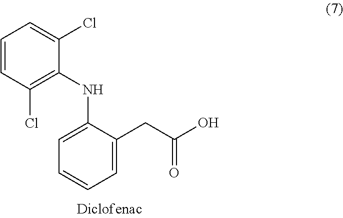 [SMILES: O=C(O)Cc1ccccc1Nc1c(Cl)cccc1Cl]

Group 4: Indoleacetic Acid Derivatives (8)

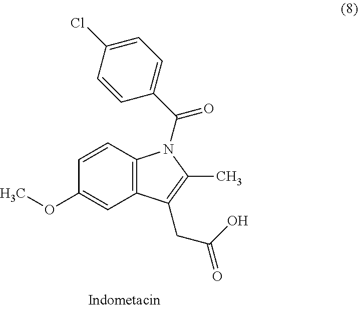 [SMILES: COc1ccc2c(c1)c(CC(=O)O)c(C)n2C(=O)c1ccc(Cl)cc1]

Group 5: Anthranilic Acid Derivatives (9) (10)

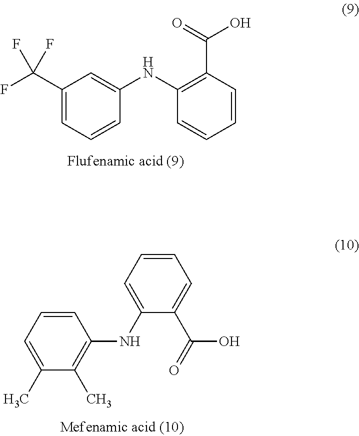 [SMILES: O=C(O)c1ccccc1Nc1cccc(C(F)(F)F)c1 ; Cc1cccc(Nc2ccccc2C(=O)O)c1C]

Group 6: Oxicams (11) (13)

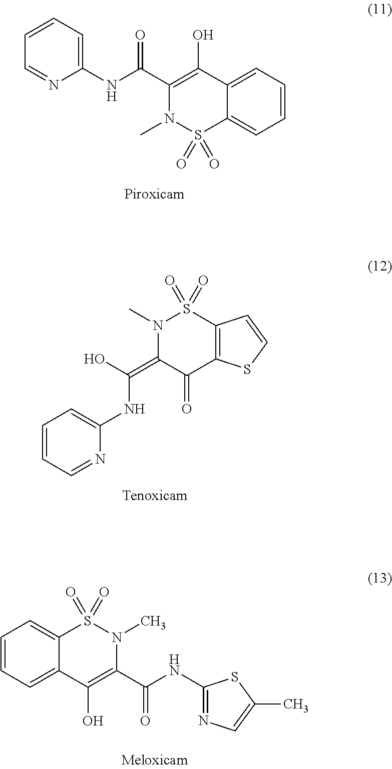 [SMILES: CN1C(C(=O)Nc2ccccn2)=C(O)c2ccccc2S1(=O)=O ; CN1/C(=C(\O)Nc2ccccn2)C(=O)c2sccc2S1(=O)=O ; Cc1cnc(NC(=O)C2=C(O)c3ccccc3S(=O)(=O)N2C)s1]

Group 7: Aminophenols

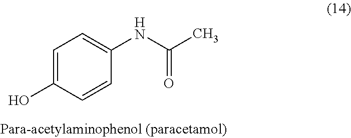

Dehydroabietic Acid

The diterpene dehydroabietic acid (component (b)) is present both in the needles and the bark, root and particularly the resin of many conifers (among others, in the plant families of Pinaceae and Juniperaceae, and here, in particular, in the genera Pinus, Abies, Larix, Juniperus). It is a by-product of paper production and contained in what is referred to as tall oil.

JP 2006 312590 A (SUNSTAR) discloses a formulation in example 7, which contains dehydroabietic acid along with methyl salicylate. U.S. Pat. No. 6,365,634 B1 (RUSSEL) discloses the use of dehydroabietic acid to inhibit the cyclooxygenase. In WO 2007 040005 A1 (UNIV KYOTO) a preparation with anti-inflammatory properties is described, containing dehydroabietic acid, but without associating the anti-inflammatory property with this substance. Eventually, the paper by Sepulveda et al. in Pharmacol. Res. Nov. 52(5), S. 429-437 (2005) reports that dehydroabietic acid has gastroprotective properties.

Dehydroabietic acid has also been isolated from other plant families (e.g., from the genera Illicium, Liquidambar, Styrax, Callicarpa, Rosmarinus, Salvia, Commiphora, Boswellia). Dehydroabietic acid is most frequently found in resins (e.g., Liquidambar, Styrax, Boswellia, Commiphora, Colophony, but also in amber).

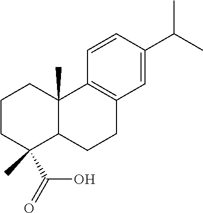 [SMILES: CC(C)c1ccc2c(c1)CCC1[C@](C)(C(=O)O)CCC[C@]21C]

Structural Formula of Dehydroabietic Acid (CAS 1740-19-8)

The content of dehydroabietic acid in pine resin is mostly below 10%, but it may also be increased to a content of >50%, using a method that is known by the skilled person as disproportionation (e.g., according to the process described by Song et al in JOURNAL OF WOOD CHEMISTRY AND TECHNOLOGY, 5(4), 535-542 (1985)).

Both enantiomers of dehydroabietic acid are known (CAS 1740-19-8 and CAS 6980-63-8). The term “dehydroabietic acid” is understood as meaning the isomers and any optional enantiomeric mixtures within the meaning of the present invention.

Although extracts containing dehydroabietic acid as well as the commercially available technical dehydroabietic acid having a purity of about 85% can be used, the use of high-purity products is preferred, which have a content of dehydroabietic acid of at least 90% by weight, preferably at least 95% by weight, and particularly preferably from about 95 to about 99% by weight. These high-purity products are obtainable by the typical work-up procedures of preparative organic chemistry so that the skilled person is not required to make an inventive step. In the following, a corresponding process is described, by way of example, in example 1.

Instead of using dehydroabietic acid itself, it is also possible to use its salts, specifically its alkali and ammonium salts, particularly the sodium salt.

The invention also comprises the use of extracts containing dehydroabietic acid, the production of which also forms part of the skilled person's tools, and which is described, by way of example, in example 2 below. Preferably, these extracts contain at least 20% by weight, more preferably at least 30% by weight, and particularly preferably from about 40 to about 60% by weight dehydroabietic acid.

In sum, the improvement of the sensory profile of ibuprofen is preferred.

Sweeteners

Suitable sweeteners or sweet-tasting additives forming facultative group (c) are, firstly, carbohydrates such as, for example, trehalose, lactose, maltose, melizitose, raffinose, palatinose, lactulose, D-fructose, D-glucose, D-galactose, L-rhamnose, D-sorbose, D-mannose, D-tagatose, D-arabinose, L-arabinose, D-ribose, D-glyceraldehyde or maltodextrin. Plant-based preparations containing these substances are also suitable, for example, on the basis of sugarbeet (Beta vulgaris ssp., sugar fractions, sugar syrup, molasses), sugar cane (Saccharum officinarum ssp., molasses, sugar cane syrup), maple syrup (Acer ssp.) or agave (agave syrup).

Suitable are also synthetic, i.e. usually enzymatically produced starch or sugar hydrolysates (invert sugar, fructose syrup) as well as natural or synthetic sweet-tasting substances, such as
Fruit concentrates (e.g., on the basis of apples or pears);
Sugar alcohols (e.g., erythritol, threitol, arabitol, ribotol, xylitol, sorbitol, mannitol, dulcitol, lactitol);
Proteins (e.g., miraculin, monellin, thaumatin, curculin, brazzein);
Sweeteners (e.g., magap, sodium cyclamate, acesulfame-K, neohesperidin dihydrochalcone, saccharine sodium salt, aspartame, superaspartame, neotame, alitame, sucralose, stevioside, rebaudioside, lugduname, carrelame, sucrononate, sucrooctate, monatin, phenylodulcin);
Sweet-tasting amino acids (e.g., glycine, D-leucine, D-threonine, D-asparagine, D-phenylalanine, D-tryptophan, L-proline);
Further sweet-tasting low-molecular substances such, e.g., hernandulcin, phyllodulcin, dihydrochalcon glycoside, glycyrrhizin, glycerrhetinic acid and its derivatives and salts, rubusosides, mogrosides
Extracts of sweet-tasting plants such as Stevia rebaudiana, Glycyrrhiza ssp. (liquorice), Lippia dulcis, Momordica grosvenori.

The sweeteners may also be plant extracts, as is described by way of example in the following.

Rebaudiosides are among the steviosides, which are the main components of the plant Stevia rebaudiana, which is also referred to as sweet weed or honey weed.

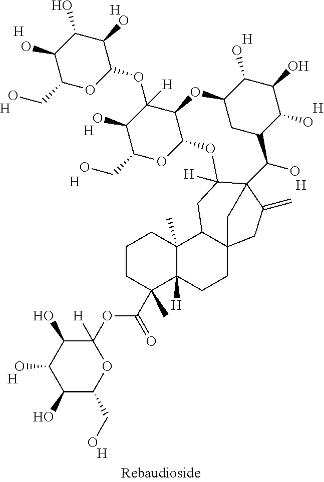

10% of the dry matter of the leaves are constituted by the diterpene glycoside stevioside, followed by rebaudioside A (2 to 4% by weight) as well as by more than ten other steviol glycosides such as dulcoside. By now, most countries have approved rebaudiosides and stevia extracts for use as sweeteners; a daily uptake of up to 4 mg stevioside per kilogramme of bodyweight is considered harmless. Within the meaning of the invention, individual rebaudiosides or the extracts of the stevia plant may be used. Particularly preferred, however, is the use of rebaudioside A, as this substance has a lower bitterness and the highest sweetening power. The substance mixtures according to the invention may contain components (a) and (b) in a weight ratio from about 1:99 to about 99:1, preferably from about 25:75 to about 75:25, and particularly preferably from about 40:60 to about 60:40.

Also the dihydrochalcones represent flavonoids, in which particularly the two representatives naringenin dihydrochalcone and neohesperidin dihydrochalcone must be highlighted, which are known as synthetic sweeteners:

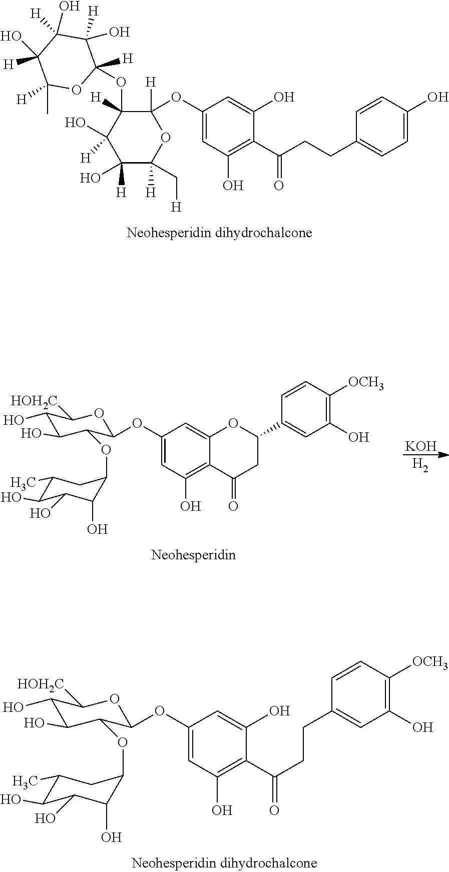 [SMILES: C[C@@H]1O[C@@H](O[C@@H]2C(Oc3cc(O)c(C(=O)CCc4ccc(O)cc4)c(O)c3)O[C@@H](C)[C@H](O)[C@H]2O)[C@H](O)[C@H](O)[C@H]1O ; COc1ccc([C@@H]2CC(=O)c3c(O)cc(OC4OC(CO)C(O)C(O)C4OC4CC(C)C(O)C(O)C4O)cc3O2)cc1O ; "KOH" ; "H2" ; COc1ccc(CCC(=O)c2c(O)cc(OC3OC(CO)C(O)C(O)C3OC3CC(C)C(O)C(O)C3O)cc2O)cc1O]

A group of cucurbitane glycosides is referred to as mogrosides, which are known as a component of the natural sweetener Luo Han Guo. Mogroside-V, which is 400 times sweeter than sugar, is highlighted herein.

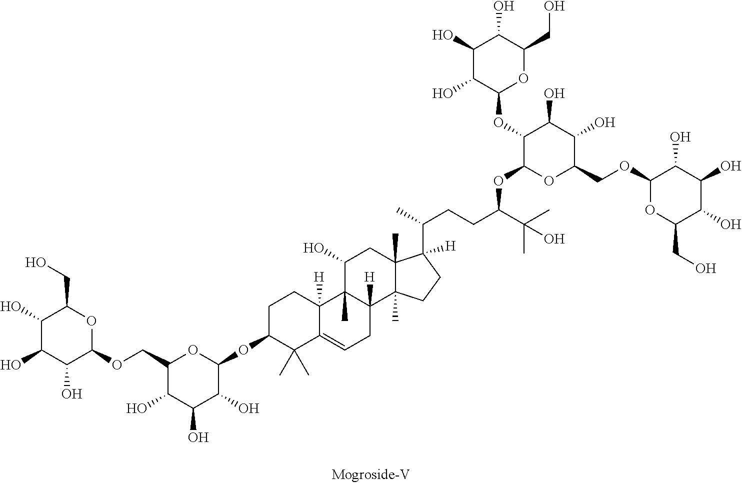

Eventually, suitable sweeteners also include extracts of the plants which are selected from the group consisting of Rubus allegheniensis, Rubus arcticu, Rubus strigosus, Rubus armeniacus, Rubus caesius, Rubus chamaemorus, Rubus corylifolius agg., Rubus fruticosus agg., Rubus geoides, Rubus glaucus, Rubus gunnianus, Rubus idaeus, Rubus illecebrosus, Rubus laciniatus, Rubus leucodermis, Rubus loganobaccus, Rubus loxensis, Rubus nepalensis, Rubus nessensis, Rubus nivalis, Rubus odoratus, Rubus pentalobus, Rubus phoenicolasius, Rubus saxatilis, Rubus setchuenensis, Rubus spectabilis and Rubus ulmifolius and their mixtures. These are substantially extracts of various blackberry and raspberry varieties having a content in rubosides. Extracts of Rubus suavissimus are preferred.

A further active agent in this group is glycyrrhetinic acid, or a corresponding salt, or an extract containing this substance.

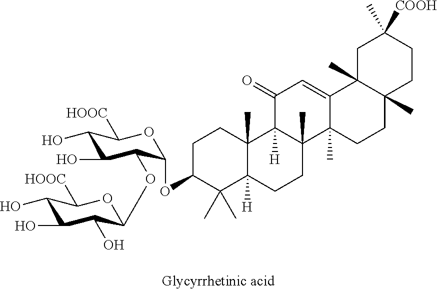

Within the meaning of the invention, it is possible to use the acid itself, its salts for example, sodium, potassium, or ammonium salt or the extracts of the plant Glycyrrhiza glabra. Mono ammonium glycyrrhizinate is particularly preferred.

Preferred sweeteners, the taste perception of which is to be improved, are selected from the group consisting of saccharin, acesulfame-K, steviol glycosides, particularly rebaudioside A, and stevia extracts.

Carriers

In a preferred embodiment, the two components (a) and (b), and, optionally, (c) are not employed individually, but are formulated with the aid of a carrier. The nature of the carrier itself is not critical, as long as it does not have an unpleasant taste of its own. For example, the carrier may be required in order to tablet the mixture. However, it can also be a pure filler or a thickener. Preferably, carbohydrates and, particularly, polysaccharides are suitable for this purpose. This includes, for example, dextrins, celluloses, more particularly microcrystalline celluloses, starches and modified starches as well as highly polymeric substances such as Xanthan Gum.

In liquid preparations, suitable carriers are toxicologically safe polyols such as glycerol or ethylene glycol, besides water and ethanol.

Preparations

The preparations according to the invention are, usually, pharmaceutical products. However, formulations are also comprised, which are over-the-counter and serve, for example, prophylactic purposes. In sum, the preparations may be present as solid or liquid formulations. Suitable solid preparations are, for example, tablets, lozenges, chewing gums or capsules. Examples of liquid formulations are, for example, syrups or sprays.

The preparations, preferably, have the following composition:
(a) about 0.01 to about 90% by weight, preferably about 0.1 to about 80% by weight, and more particularly about 1 to about 50% by weight NSAID active ingredients;
(b) about 0.001 to about 5% by weight, preferably about 0.005 to about 2% by weight and more particularly about 0.01 to about 1% by weight dehydroabietic acid or a corresponding extract;
(c) 0 to about 15, preferably about 1 to about 8% by weight, and more particularly about 2 to about 5% by weight % by weight sweeteners;
(d) 0 to about 95% by weight, preferably 10 to about 90% by weight and more particularly about 25 to about 60% by weight carriers;
   with the proviso that all quantities, optionally with further additives and adjuvants, add up to 100% by weight.

Preparations with sweeteners such as polyols (e.g., sorbitol) or sugars usually contain between 5 and 20% by weight sweeteners, preparations with so-called “High-Intensity-Sweeteners (HIS)” (e. g., saccharin or cyclamate), in contrast, contain less than 1% by weight sweetener. Often, mixtures of different sweeteners are used to minimize the unwelcome, frequently metallic or bitter aftertaste.

Suitable further additives and adjuvants are, particularly, colours and aromas.

Colours

Food colours or, shortly, colours are food additives for colouring preparations suitable for consumption. Colours are classified into the groups of natural colours and synthetic colours. The nature-identical colours are also of synthetic origin. The nature-identical colours are synthetic reproductions of colouring substances that occur in nature. Suitable colours for use in the present composition are selected from: Curcumin (E 100), Riboflavin, Lactoflavin, Vitamin B2 (E 101), Tartrazin (E 102), Quinoline Yellow (E 104), orange yellow S, sunset yellow RGL (E 110), Cochineal, Carminic acid, Carmines (E 120), Azorubine, Carmoisine (E 122), Amaranth (E 123), Cochineal Red A, Ponceau 4 R, Brilliant Scarlet 4 R (E 124), Erythrosine (E 127), Allura Red AC (E 129), Patent Blue V (E 131), Indigotine, Indigo Carmine (E 132), Brilliant Blue FCF, Patent Blue AE, Amido Blue AE (E 133), Chlorophylls, Chlorophyllins (E 140), Copper complexes of chlorophyll and chlorophyllins (E 141), Acid Green, Green S (E 142), Plain caramel (E 150 a), Caustic sulphite caramel (E 150 b), Ammonia caramel (E 150 c), Sulphite ammonia caramel (E 150 d), Brilliant black FCF, Brilliant black PN, Black PN (E 151), Vegetable carbon (E 153), Brown FK (E 154), Brown HT (E 155), Carotenes (E 160 a), Annatto, Bixin, Norbixin (E 160 b), Capsanthian, Capsorubin (E 160 c), Lycopene (E 160 d), Beta-apo-8′-Carotenal (C30), Apocarotenal, Beta-Apocarotenal (E 160 e), Apocarotenal ester, Beta-Carotenal acid ester (E 160 f), Lutein, Xanthophyll (E 161 b), Canthaxanthin (E 161 g), Betanin, Beetroot Red (E 162), Anthocyanins (E 163), Calcium carbonate (E 170), Titanium dioxide (E 171), Iron oxides and hydroxides (E 172), Aluminium (E 173), Silver (E 174), Gold (E 175), Litholrubine BK, Rubin pigment BK (E 180).

Aroma Substances

The preparations according to the invention may contain one or more aroma substances. Typical examples comprise: acetophenone, allyl caproate, alpha-ionone, beta-ionone, aniseed aldehyde, anisyl acetate, anisyl formate, benzaldehyde, benzothiazole, benzyl acetate, benzyl alcohol, benzyl benzoate, beta-ionone, butyl butyrate, butyl capronate, butylidene phthalide, carvone, camphene, caryophyllene, cineol, cinnamyl acetate, citral, citronellol, citronellal, citronellyl acetate, cyclohexyl acetate, cymol, damascone, decalactone, dihydrocoumarin, dimethyl anthranilate, dimethyl anthranilate, dodecalactone, ethoxy ethyl acetate, ethylbutyric acid, ethyl butyrate, ethyl caprinate, ethyl capronate, ethyl crotonate, ethyl furaneol, ethylguaiakol, ethyl isobutyrate, ethyl isovalerianate, ethyl lactate, ethylmethyl butyrate, ethyl propionate, eucalyptol, eugenol, ethyl heptylate, 4-(p-hydroxyphenyl)-2-butanone, gamma-decalactone, geraniol, geranyl acetate, geranyl acetate, grapefruit aldehyde, methyl dihydrojasmonate (e.g., 5 Hedion®), heliotropin, 2-heptanone, 3-heptanone, 4-heptanone, trans-2-heptenal, cis-4-heptenal, trans-2-hexenal, cis-3-hexenol, trans-2-hexenoic acid, trans-3-hexenoic acid, cis-2-hexenyl acetate, cis-3-hexenyl acetate, cis-3-hexenyl capronate, trans-2-hexenyl capronate, cis-3-hexenyl formiate, cis-2-hexyl acetate, cis-3-hexyl acetate, trans-2-hexyl acetate, cis-3-hexyl formiate, para-hydroxybenzyl acetone, isoamyl alcohol, isoamyl isovalerianate, isobutyl butyrate, isobutyl aldehyde, isoeugenol methyl ether, isopropyl methyl thiazole, lauric acid, levulinic acid, linalool, linalool oxide, linalyl acetate, menthol, menthofuran, methyl anthranilate, methyl butanol, methyl butyric acid, 2-methyl butyl acetate, methyl capronate, methyl cinnamate, 5-methyl furfural, 3,2,2-methyl cyclopentenolone, 6,5,2-methyl heptenone, methyl dihydrojasmonate, methyljasmonate, 2-methyl methylbutyrate, 2-methyl-2-pentanoic acid, methyl thiobutyrate, 3,1-methyl thiohexanol, 3-methyl thiohexyl acetate, nerol, neryl acetate, trans,trans-2,4-nonadienal, 2,4-nonadienol, 2,6-nonadienol, 2,4-nonadienol, nootkatone, delta octalactone, gamma octalactone, 2-octanol, 3-octanol, 1,3-octenol, 1-octyl acetate, 3-octyl acetate, palmitic acid, paraldehyde, phellandrene, pentandione, phenylethyl acetate, phenylethyl alcohol, phenylethyl alcohol, phenylethyl isovalerianate, piperonal, propionaldehyde, propyl butyrate, pulegon, pulegol, sinensal, sulfurol, terpinene, terpineol, terpinolene, 8,3-thiomenthanone, 4,4,2-thiomethyl pentanone, thymol, delta-undecalactone, gamma-undecalactone, valencene, valeric acid, vanilline, acetoine, ethyl vanilline, ethyl vanilline isobutyrate (=3-ethoxy-4-isobutyryloxybenzaldehyde), 2,5-dimethyl-4-hydroxy-3(2H)-furanone and derivatives (here, preferably homofuraneol (=2-ethyl-4-hydroxy-5-methyl-3(2H)-furanone), homofuronol (=2-ethyl-5-methyl-4-hydroxy-3(2H)-furanone and 5-ethyl-2-methyl-4-hydroxy-3(2H)-furanone), maltol and maltol derivatives (here, preferably ethyl maltol), coumarine and coumarine derivatives, gamma-lactones (here, preferably gamma-undecalactone, gamma-nonalactone, gamma-decalactone), delta-lactones (here, preferably 4-methyldeltadecalactone, massoilactone, delta-decalactone, tubero lactone), methyl sorbate, divanilline, 4-hydroxy-2(or 5)-ethyl-5(or 2)-methyl-3(2H)furanone, 2-hydroxy-3-methyl-2-cyclopentenon, 3-hydroxy-4,5-dimethyl-2(5H)furanone, isoamyl acetate, ethyl butyrate, butyl butyrate, isoamyl butyrate, methyl-3-ethyl butyrate, n-hexanoic acid allyl ester, n-hexanoic acid-n-butyl ester, n-ethyl octanoate, ethyl-3-methyl-3-phenylglycidate, ethyl-2-trans-4-cis-decadienoate, 4-(p-hydroxyphenyl)-2-butanone, 1,1-dimethoxy-2,2,5-trimethyl-4-hexane, 2,6-dimethyl-5-hepten-1-al and phenylacetaldehyde, 2-methyl-3-(methylthio)furane, 2-methyl-3-furanthiol, bis(2-methyl-3-furyl)disulfide, furfuryl mercaptane, methional, 2-acetyl-2-thiazoline, 3-mercapto-2-pentanone, 2,5-dimethyl-3-furanthiol, 2,4,5-trimethylthiazol, 2-acetylthiazol, 2,4-dimethyl-5-ethylthiazol, 2-acetyl-1-pyrroline, 2-methyl-3-ethylpyrazine, 2-ethyl-3,5-dimethylpyrazine, 2-ethyl-3,6-dimethylpyrazine, 2,3-diethyl-5-methylpyrazine, 3-isopropyl-2-methoxypyrazine, 3-Isobutyl-2-methoxypyrazine, 2-acetylpyrazine, 2-pentylpyridine, (E,E)-2,4-decadienal, (E,E)-2,4-nonadienal, (E)-2-octenal, (E)-2-nonenal, 2-undecenal, 12-methyltridecanal, 1-Penten-3-one, 4-hydroxy-2,5-dimethyl-3(2H)-furanone, guaiakol, 3-hydroxy-4,5-dimethyl-2(5H)-furanone, 3-hydroxy-4-methyl-5-ethyl-2(5H)-furanone, cinnamon aldehyde, cinnamon alcohol, methyl salicylate, isopulegol and (not explicitly mentioned here) stereoisomers, enantiomers, positional isomers, diastereomers, cis/trans-isomers and epimers of these substances.

In the following, a number of galenic forms in which the preparations according to the invention may be present will be explained in more detail.

Tablets

Tablets, containing or consisting of the preparations according to the invention, may contain components that are typical for this form of administration, as there are, for example: carriers (as already explained above), disintegrants and/or colours and aroma substances. Tabletting itself is a sufficiently known industrial method. By way of example, it is referred to DE 10 2006 051529 A1 (HENKEL) where the production of tablets is explained in more detail.

Carriers

Suitable carrier may be classified in filling agents and binding agents. Fillers ensure that the tablet is of the required size/mass. Starches (maize starch, potato starch and wheat starch) and lactose are employed. Other fillers are glucose, mannitol, sorbitol. Due to its high price, fructose is only very rarely used. Saccharose is mainly used for lozenges.

In contrast to this, binders ensure the cohesion of the granules or powders and the solidity of tablets, besides the application of pressure. They are divided into dry binders such as, e.g. MCC (microcrystalline cellulose) or starch, and into solution binders/adhesives for granulation such as, e.g., starch pastes, cellulose ether, Kollidon and gelatin.

Disintegrants

To facilitate the disintegration of prefabricated moulded bodies it is possible to incorporate disintegration adjuvants, so-called tablet disintegrants, into these means in order to shorten disintegration times. Tablet disintegrants are understood to be adjuvants that ensure a quick disintegration of tablets in water or other media, and a fast release of the active ingredients.

These substances, which are referred to as disintegrants as a result of their effect, increase their volume when upon contact with water, in the process of which their own volume increases (swelling) on the one hand, and pressure allowing the tablet to disintegrate into smaller particles may be generated through the release of gases on the other. Well-known disintegration aids are, for example, carbonate-citric acid systems, however, other organic acids may be used as well. Swelling disintegration aids are, for example, synthetic polymers such as polyvinyl pyrrolidone (PVP) or natural polymers or modified natural substances such as cellulose and starch and the derivatives thereof, alginates, or casein derivatives.

Preferred disintegration means are disintegration means on the basis of cellulose. Pure cellulose has the formal overall composition (C₆H₁₀O₅)ₙ and represents, formally, a beta-1,4-polyacetate of cellobiose, which itself is composed of two glucose molecules. In this context, suitable celluloses consist of about 500 to 5000 glucose units, thus having average molecular masses of 50,000 to 500,000. Within the scope of the present invention, suitable disintegration means on the basis of cellulose are also cellulose derivatives that are obtainable from cellulose by polymer-analogous reactions. For example, these chemically modified celluloses comprise products from esterifications or etherifications, in which hydroxy-hydrogen atoms were substituted. However, also celluloses, in which the hydroxy groups were replaced by functional groups that are not bound via an oxygen atom, may be employed as cellulose derivatives. The group of cellulose derivatives includes, for example, alkali celluloses, carboxymethyl cellulose (CMC), cellulose esters, cellulose ethers, and aminocelluloses. The above cellulose derivatives are, preferably, not solely used as disintegration means on the basis of cellulose, but are used in mixture with cellulose. The content of cellulose derivatives of these mixtures, preferably, is below 50% by weight, more particularly, below 20% by weight, based on the disintegration means on the basis of cellulose. Pure cellulose is particularly preferably used as disintegration means on the basis of cellulose, which is free of cellulose derivatives.

The cellulose used as disintegration means, preferably, is not used in the form of fine particles but is transformed into a coarser form, for example, granulated or compacted, before adding it to the pre-mixtures that are to be compressed. Particle sizes of such disintegration means are mostly above 200 μm, preferably, to at least 90% by weight between 300 and 1,600 μm, and particularly preferably, to at least 90% by weight between 400 and 1,200 μm.

Microcrystalline cellulose may be employed as a further disintegration means on the basis of cellulose or as a part of this component. This microcrystalline cellulose is obtained by partial hydrolysis of celluloses under those conditions which only tackle the amorphous regions (ca. 30% of the total mass of the cellulose) of the celluloses, completely dissolving them, however, leaving the crystalline regions (ca. 70%) undamaged. A subsequent disaggregation of the microfine celluloses that were formed by hydrolysis, supplies the microcrystal line celluloses which have primary particle sizes of ca. 5 μm and may be compacted, for example, to form granules with a mean particle size of 200 μm.

Moreover, gas-developing effervescent systems may be employed advantageously according to the invention. The gas-developing effervescent system may consist of a single substance which releases a gas upon contact with water. Among these compounds, particularly magnesium peroxide needs to be mentioned, which releases oxygen upon contact with water. However, the gas-releasing effervescent system itself usually consists of at least two components which react with one another, forming gas. While herein a multitude of systems is both conceivable and feasible, which release, for example, nitrogen, oxygen or hydrogen, the effervescent system employed in detergents and cleaning agents may be selected both for economic and ecologic aspects. Preferred effervescent systems consist of alkali metal carbonate and/or alkali hydrogen carbonate as well as an acidification means, which is suitable for releasing carbon dioxide from the alkali metal salts in an aqueous solution.

Chewing Gums

The preparations according to the invention may also be chewing gums. These products typically contain a water-insoluble and a water-soluble component.

The water-insoluble base, which is also known as “gum base”, typically comprises natural or synthetic elastomers, resins, fats and oils, plasticizers and softeners, fillers, colourants and optionally waxes. The base normally makes up 5 to 95% by weight, preferably 10 to 50% by weight, and more particularly 20 to 35% by weight of the composition as a whole. In a typical form of embodiment of the invention, the base consists of 20 to 60% by weight synthetic elastomers, 0 to 30% by weight natural elastomers, 5 to 55% by weight plasticizers, 4 to 35% by weight fillers, and small quantities of additives such as colourants, antioxidants and the like, with the proviso that they are soluble in water in small quantities at best.

Suitable synthetic elastomers are, for example, polyisobutylenes with average molecular weights (as measured by GPC) of 10,000 to 100,000 and preferably 50,000 to 80,000, isobutylen/isoprene copolymers (“butyl elastomers”), styrene/butadiene copolymers (styrene:butadiene ratio, for example, 1:3 to 3:1), polyvinyl acetates with average molecular weights (as measured by GPC) of 2,000 to 90,000 and preferably 10,000 to 65,000, polyisoprenes, poly-ethylenes, vinyl acetate/vinyl laurate copolymers and mixtures thereof. Examples of suitable natural elastomers are rubbers such as, for example, smoked or liquid latex or guayuls, and natural gums, such as jelutong, lechi caspi, perillo, sorva, massaranduba balata, massaranduba chocolate, nispero, rosindinba, chicle, gutta hang kang and mixtures thereof. The choice of the synthetic and natural elastomers and their mixing ratios essentially depends on whether bubbles are to be produced with the chewing gums (“bubble gums”) or not. Elastomer mixtures containing jelutong, chicle, sorva and massanduraba are preferably used.

In most cases, the elastomers are too hard or lack plasticity for satisfactory processing, so it has been found to be of advantage to use special plasticizers which, of course, must also satisfy in particular all requirements relating to acceptability as food additives. In this respect, suitable plasticizers are, above all, esters of resin acids, for example, esters of lower aliphatic alcohols or polyols with completely or partly hydrogenated, monomeric or oligomeric resin acids. In particular, the methyl, glycerol or pentaerythritol esters or mixtures thereof are used for this purpose. Alternatively, terpene resins, which may be derived from α-pinene, β-pinene, δ-limonene or mixtures thereof, could also be used.

Suitable fillers or texturizers are magnesium or calcium carbonate, ground pumice stone, silicates, especially magnesium or aluminium silicates, clays, aluminium oxides, talcum, titanium dioxide, mono-, di- and tricalcium phosphate and cellulose polymers.

Suitable emulsifiers are tallow, hydrogenated tallow, hydrogenated or partly hydrogenated vegetable oils, cocoa butter, partial glycerides, lecithin, triacetin and saturated or unsaturated fatty acids containing 6 to 22 and preferably 12 to 18 carbon atoms and mixtures thereof.

Suitable colourants and whiteners are, for example, the FD & C types, plant and fruit extracts permitted for colouring foods and titanium dioxide.

The gum bases may contain waxes, or may be wax-free; examples of wax-free compositions can be found inter alia in U.S. Pat. No. 5,286,500, to the disclosure of which reference is hereby specifically made. In addition to the water-insoluble gum base, chewing gum compositions regularly contain a water-soluble component, which is formed, for example, by softeners, sweeteners, fillers, flavours, flavour enhancers, emulsifiers, colourants, acidifiers, antioxidants and the like, with the proviso in this case that the constituents have at least adequate solubility in water. Accordingly, individual constituents may belong both to the water-insoluble phase and to the water-soluble phase, depending on the water solubility of the special representatives. However, combinations may also be used, for example, a combination of a water-soluble and a water-insoluble emulsifier, in which case the individual representatives are present in different phases. The water-insoluble component usually makes up 5 to 95% by weight and preferably 20 to 80% by weight of the preparation.

Water-soluble softeners or plasticizers are added to the chewing gum compositions to improve chewability and the chewing feel and are present in the mixtures in quantities of typically 0.5 to 15% by weight. Typical examples are glycerol, lecithin and aqueous solutions of sorbitol, hydrogenated starch hydrolysates or maize syrup.

Suitable sweeteners are both sugar-containing or sugar-free compounds which are used in quantities of 5 to 95% by weight, preferably in quantities of 20 to 80% b weight and more particularly in quantities of 30 to 60% by weight, based on the chewing gum composition. Typical saccha ride sweeteners are sucrose, dextrose, maltose, dextrin, dried invert sugar, fructose, fructose, levulose, galactose, maize syrup and mixtures thereof. Suitable sugar substitutes are sorbitol, mannitol, xylitol, hydrogenated strarch hydrolysates, maltitol and mixtures thereof. Further suitable additives are so-called high-intensity artificial sweeteners (HIAS) such as, for example, sucralose, aspartame, acesulfam salts, alitam, saccharin and saccharin salts, cyclamic acid and salts thereof, glycyrrhicins, dihydrochalcones, thaumatin, monellin and the like either individually or in the form of mixtures. Particularly effective are also the hydrophobic HIAS which are the subject matter of international patent application WO 2002 091849 A1 (Wrigleys), as well as Stevia extracts and the active components thereof, particularly, ribeaudioside A. The applied quantity of these substances primarily depends on their performance and is typically in the range of 0.02 to 8% by weight.

Fillers are particularly suitable for the production of low-calorie chewing gums and may be selected, for example, from polydextrose, raftilose, raftilin, fructo-oligosaccharides (Nutra-Flora), palatinose oligosaaccharides, guar gum hydrolysates (Sun Fiber) and dextrins.

The choice of other flavours is virtually unlimited and is not critical to the essence of the invention. All flavours normally make up from 0.1 to 15% by weight and, preferably, from 0.2 to 5% by weight of the chewing gum composition. Suitable flavours are, for example, essential oils, synthetic aromas and the like, such as, for example, aniseed oil, Japanese anise oil, caraway oil, eucalyptus oil, fennel oil, citrus oil, wintergreen oil, clove oil, menthol and the like, such as used, for example, in oral and dental care products.

Capsules

Capsules that may contain the preparations according to the invention are understood to be spherical aggregates which contain at least one solid or liquid core surrounded by at least one continuous membrane. The active agents may be encapsulated by means of coating materials and be present as macrocapsules with a diameter of about 0.1 to about 5 mm or as microcapsules with a diameter of about 0.0001 to about 0.1 mm.

Coating Materials

Suitable coating materials in this case are, for example, starches, including their degradation products as well as chemically or physically produced derivatives (more particularly, dextrins and maltodextrins) and mixtures of two or more of the following substances: gelatin, gum arabic, agar-agar, ghatti gum, gellan gum, modified and non-modified celluloses, pullulan, curdlan, carrageenan, alginic acid, pectin, inulin, xanthan gum.

The solid encapsulating material is, preferably, a gelatin (more particularly, porcine, bovine, poultry and/or fish gelatin), which, preferably, has a swelling factor of more than or equal to 20, more particularly, of more than or equal to 24. Among these substances, gelatin is particularly preferred, as it is readily available and can be purchased with different swelling factors.

Also preferred are maltodextrins (more particularly, on the basis of cereal, specifically maize, wheat, tapioca or potatoes), which preferably have DE values within the range of 10 to 20. Also preferred are celluloses (for example, cellulose ethers), alginates (for example, sodium alginate), carrageenan (for example, beta, iota, lambda and/or kappa carrageenan), gum arabic, curdlan and/or agar agar).

Alginate capsules are also preferred, as they are disclosed in detail, for example, in EP 0389700 A1, U.S. Pat. No. 4,251,195, U.S. Pat. No. 6,214,376, WO 2003 055587 or WO 2004 050069 A1.

In another preferred embodiment, the membrane of the capsules consists of melamine formaldehyde resins or of coacervation products of cationic monomers or biopolymers (such as, e.g., chitosan) and anionic monomers such as, for example (meth)acrylates or alginates.

Encapsulation Methods

Generally, capsules are finely dispersed liquid or solid phases coated with film-forming polymers, in the production of which the polymers are deposited onto the material to be encapsulated after emulsification and coacervation or interfacial polymerization. In another process, molten waxes are absorbed in a matrix (“microsponge”) which, as microparticles, may be additionally coated with film-forming polymers. According to a third process, particles are alternatingly coated with polyelectrolytes of different charges (“layer-by-layer” method). The microscopically small capsules can be dried in the same way as powders. Besides single-core microcapsules, there are also multiple-core aggregates, also known as microspheres, which contain two or more cores distributed in the continuous membrane material. In addition, single-core or multiple-core microcapsules may be surrounded by an additional second, third etc. membrane. The membrane may consist of natural, semisynthetic or synthetic materials. Natural membrane materials are, for example, gum arabic, agar agar, agarose, maltodextrins, alginic acid and salts thereof, for example, sodium or calcium alginate, fats and fatty acids, cetyl alcohol, collagen, chitosan, lecithins, gelatin, albumin, shellac, polysaccharides such as starch or dextran, polypeptides, protein hydrolyzates, sucrose and waxes. Semisynthetic membrane materials are inter alia chemically modified celluloses, more particularly cellulose esters and ethers, for example, cellulose acetate, ethyl cellulose, hydroxypropyl cellulose, hydroxypropyl methyl cellulose and carboxymethyl cellulose, and starch derivatives, more particularly starch ethers and esters. Synthetic membrane materials are, for example, polymers such as polyacrylate, polyamide, polyvinyl alcohol or polyvinyl pyrrolidone.

Examples of state of the art microcapsules are the following commercial products (the membrane material is shown in brackets) Hallcrest Microcapsules (gelatin, gum arabic), Coletica Thalaspheres (maritime collagen), Lipotec Millicapseln (alginic acid, agar agar), Induchem Unispheres (lactose, microcrystalline cellulose, hydroxypropylnnethyl cellulose), Unicerin C30 (lactose, microcrystalline cellulose, hyd roxypropyl methyl cellulose), Kobo Glycospheres (modified starch, fatty acid esters, phospholipids), Softspheres (modified agar agar) and Kuhs Probiol Nanospheres (phospholipids) as well as Primaspheres and Primasponges (chitosan, alginates) and Primasys (phospholipids).

Microcapsules made of chitosan and processes for their production are well known from the state of the art [WO 01/01926, WO 01/01927, WO 01/01928, WO 01/01929]. Microcapsules having mean diameters in the range from 0.0001 to 5, preferably 0.001 to 0.5 and particularly preferably 0.005 to 0.1 mm, consisting of a coating membrane and a matrix containing the active agents, may be obtained, for example, by

(a) preparing a matrix composed of gel formers, cationic polymers and active agents,
(b) optionally, dispersing the matrix in an oil phase,
(c) treating the dispersed matrix with aqueous solutions of anionic polymers, optionally removing the oil phase in this process.

Here, steps (a) and (c) are exchangeable insofar that anionic polymers are used instead of the cationic polymers, and vice versa.

It is also possible to produce the capsules by coating the capsules in alternating layers of differently charged polyelectrolytes (layer-by-layer method). In this context, it is referred to the European patent EP 1064088 B1 (Max-Planck-Gesellschaft).

### INDUSTRIAL APPLICABILITY

Further subject matters of the present invention, on the one hand, relate to
a process for masking the unpleasant taste of NSAID active ingredients and formulations containing these active ingredients, and
a process for alleviating and/or reducing the burning sensation of NSAID active ingredients and of formulations containing these active ingredients in the throat and on the tongue, and for an increase of mouth moisture on the other,
   wherein the active ingredients are formulated along with dehydroabietic acid, or an extract containing dehydroabietic acid, and are then administered orally.

Eventually, the invention also comprises the use of dehydroabietic acid, or extracts containing dehydroabietic acid, for
(i) masking the bitter and/or metallic taste of NSAID active ingredients and, optionally, further formulation components;
(ii) alleviating or reducing the burning sensation of NSAID active ingredients and of formulations containing these active ingredients in the throat and on the tongue;
(iii) increasing mouth moisture;
(iv) intensifying the aromas and the sweetness of formulations containing NSAID active ingredients;
(v) improving the mucous membrane compatibility during oral uptake of formulations containing NSAID active ingredients.

These indications apply to the claimed processes and the use, mutatis mutandis, without requiring further mentioning or repeating, as far as any preferred embodiments, specifically, particularly preferred NSAID, NSAID formulations, aids, quantities and ratios of quantities have been explained above.

In the following, the invention is illustrated by means of a series of exemplary embodiments, however, without limiting it to those.

### EXAMPLES

### Examples of Production

### Example H1

Purification of Dehydroabietic Acid

2 g of commercially available dehydroabietic acid (CAS 1740-19-8, purchased from Interchim, 211 bis AVENUE KENNEDY, BP 1140, 03103 MONTLUCON CEDEX, France) with a content of ca. 85% was purified by preparative HPLC chromatography as follows.

Separation number: H-2074-B

Stationary phase: LichrospherSelect B, 10 unn, 250×50 mm

Mobile phase A: 5 mMol ammonium formate buffer, set to pH 3.0 with formic acid

Mobile phase B: methanol acetonitril 1:1 (v/v) with 5 mMol ammonium formate

Gradient: from 62% to 81% B in 57 min

Flow rate: 80 ml/min

Detection: ELSD

Fractions containing the product were combined, the solvent was evaporated in a vacuum, and the isolated dehydroabietic acid was analytically characterised by means of H-NMR spectroscopy and LC-MS. The identity of the isolated dehydroabietic acid was confirmed by the NMR and the molecular mass, the purity was at >98%.

The LC-MS method for analytical characterisation of the isolated substance is summarised in the following Table 1:

TABLE H1
LC-MS method

| HPLC system | PE Series 200 | | |
|---|---|---|---|
| MS system | Applied Biosystems API 150 | | |
| Data system | Analyst 1.3 | | |
| Stationary phase | Merck Select B 250 × 4 mm, 5 μm | | |
| Flow rate | 1 ml/min | | |
| Detection | (+/(−)-ESI, Fast-Switching-Mode | | |
| | ELSD (Sedex 75) | | |
| | UV (Merck, 254 nm) | | |
| Sample | 10 mg/ml in DMSO | | |
| concentration | | | |
| Injection volume | 30 μl | | |
| Mobile phase: | A: 5 mM ammonium formate and 0.1% formic acid | | |
| | B: acetonitrile/methanol = 1:1, 5 mM | | |
| | ammonium formate and 0.1% formic acid (pH 3) | | |
| Gradient | Time [min] | % A | % B |
| | 00.0 | 85 | 15.00 |
| | 30.0 | 0 | 100.0 |
| | 35.0 | 0 | 100.0 |

### Example H2

Production of an Extract Containing Dehydroabietic Acid

10 g commercially available colophony resin (purchased from Alfred Galke GmbH, Am Bahnhof 1, 37539 Bad Grund, order number 36004) is extracted with a mixture of MTB ether and methanol (1:1 v/v), removing the solvent in a vacuum. The extract contains ca. 25% dehydroabietic acid.

### Application-Related Examples

The following Tables A and B list ingredients of the tested formulations (without the respective active agent according to the examples):

TABLE A
Ingredients I

| Preservation agents | Sweeteners | Aromas | Adjuvants |
|---|---|---|---|
| Methyl-parahydroxybenzoate | Sorbitol (E420) | Strawberry | Glycerol |
| (E218, 219) | Maltitol (E965) | Orange | Acetylated monoglycerides |
| Propyl-parahydroxybenzoate | Acesulfame K | | Propylene glycol |
| (E216, 217) | Saccharin | | Mg-Stearate |
| Methylparaben | Sucrose | | Na-Laurylsulfate |
| Propylparaben | | | Stearic acid |
| Na-Metabisulphite (E223) | | | French chalk (silicate) |
| Domiphen bromide | | | Povidone |
| Sodium benzoate | | | Polysorbate 80 |
| | | | Na-Starch glycollate Type A |
| | | | Croscarmellose sodium |
| | | | Colloidal silicon dioxide |

TABLE B
Ingredients II

| Fillers/Thickeners | Colourants/Coatings |
|---|---|
| Microcrystalline cellulose | Colloidal silicone dioxide (E171) |
| Maize starch | Opaspray White M-1-7111B (contains |
| Xanthan gum | hypromellose, titanium dioxide E171) |
| Dispersible cellulose | Black ink (contains shellac, iron oxide |
| | black E172) |
| Hypromellose | Pharmaceutical ink |
| Pregelatinized starch | Pharmaceutical glaze |
| | Synthetic iron oxide |
| | White wax |

### Example 1

Taste Modulation of Paracetamol in a Commercially Available Formulation

The sensory evaluation of the samples was performed by a team of five experienced assessors. Details may be taken from the following Table 1:

TABLE 1
Tasting of a Calpol ® solution

| Test method | Descriptive and discriminative | |
|---|---|---|
| | evaluation, “sip and spit” | |
| | method, blinded and randomised samples, | |
| Materials | Purified dehydroabietic acid, purity > 98% | |
| | Calpol ®: sugar-free suspension with | |
| | strawberry taste, sweetened with acesulfame | |
| | K and sodium saccharin | |
| Panelists | 5 experienced assessors | |
| Preparation | (A) | Dehydroabietic acid, dissolved in |
| of the samples | | ethanol |
| | (B) | Calpol ® suspension in water, |
| | | final concentration of paracetamol |
| | | was 12.5 mg/ml |

| | |
|---|---|
| | Sample: (A) + (B), final 25 μM |
| | dehydroabietic acid, 0.5% ethanol |
| | Comparison sample: (B) incl. 0.5% ethanol |
| Evaluation of the sample | very significantly less bitter |
| Calpol ® suspension in | metallic taste reduced |
| water plus 25 μM | |
| dehydroabietic acid | |

### Example 2

Taste Modulation of Paracetamol in a Maltitol Formulation

The sensory evaluation of the samples was performed by a team of five experienced assessors. Details may be taken from the following Table 2:

TABLE 2
Tasting of a maltitol/paracetamol solution

| Test method | Descriptive and discriminative | |
|---|---|---|
| | evaluation, “sip and spit” | |
| | method, blinded and randomised samples, | |
| Test sample | Purified dehydroabietic acid, purity > 98% | |
| | Suspension of paracetamol dissolved in | |
| | maltitol syrup | |
| Panelists | 5 experienced assessors | |
| Preparation | (A) | Dehydroabietic acid dissolved in |
| of the samples | | ethanol |
| | (B) | Paracetamol/maltitol syrup dissolved in |
| | | water, final concentration of |
| | | paracetamol was 12.5 mg/ml |

| | |
|---|---|
| | Sample: (A) + (B), final 25 μM |
| | dehydroabietic acid, 0.5% ethanol |
| | Comparison sample: (B) incl. 0.5% ethanol |
| Evaluation of the sample | Bitterness reduced and delayed |
| Paracetamol/maltitol | no long-lasting unpleasant/metallic taste |
| syrup in water plus 25 μM | better taste at the beginning and more |
| dehydroabietic acid | pleasant aftertaste |

### Example 3

Taste Modulation of Ibuprofen in the Form of Tablets with Lysine (Nurofen®)

The sensory evaluation of the samples was performed by a team of five experienced assessors. Details may be taken from the following Table 3:

TABLE 3
Tasting of a Nurofen ® solution

| Test method | Descriptive and discriminative | |
|---|---|---|
| | evaluation, “sip and spit” | |
| | method, blinded and randomised samples | |
| Test sample | Purified dehydroabietic acid, purity > 98% | |
| | Suspension of Nurofen ® tablets in water | |
| Panelists | 5 experienced assessors | |
| Preparation | (A) | Dehydroabietic acid, dissolved in |
| of the samples | | ethanol |
| | (B) | Nurofen ® tablets, dissolved |
| | | in water, final concentration |
| | | of ibuprofen was 5 mg/ml |

| | |
|---|---|
| | Sample: (A) + (B), final 25 μM |
| | dehydroabietic acid, 0.5% ethanol |
| | Comparison sample: Nurofen ® tablets, |
| | dissolved in water with 0.5% ethanol |
| Evaluation of the sample | reduced bitterness |
| Nurofen ® tablets, | no change to the delayed burning |
| dissolved in water plus 25 μM | sensation in the throat |
| dehydroabietic acid | |

### Example 4

Taste Modulation of Ibuprofen in Suspension (Nurofen®)

The sensory evaluation of the samples was performed by a team of five experienced assessors. Details may be taken from the following Table 4:

TABLE 4
Tasting of a solution of a Nurofen ® suspension

| Test method | Descriptive and discriminative | |
|---|---|---|
| | evaluation, “sip and spit” | |
| | method, blinded and randomised samples | |
| Test sample | Purified dehydroabietic acid, purity > 98% | |
| | Nurofen ®: sugar-free suspension | |
| | with strawberry aroma, sweetened | |
| | with sodium saccharin | |
| Panelists | 5 experienced assessors | |
| Preparation | (A) | Dehydroabietic acid, dissolved in |
| of the samples | | ethanol |
| | (B) | Nurofen ® suspension, dissolved |
| | | in water, final concentration |
| | | of ibuprofen was 5 mg/ml |
| | Sample: (A) + (B), final 25 μM | |
| | dehydroabietic acid, 0.5% ethanol | |
| | Comparison sample: Nurofen ® suspension, | |
| | dissolved in water with 0.5% ethanol | |
| Evaluation of the sample | Masking of the typical natural taste | |
| Nurofen ® suspension, | of ibuprofen. | |
| dissolved in water plus | | |
| 25 μM dehydroabietic | | |
| acid | | |

### Example 5

Taste Modulation of Ibuprofen in Suspension (Asda)

The sensory evaluation of the samples was performed by a team of five experienced assessors. Details may be taken from the following Table 5:

TABLE 5
Tasting of a solution of an ibuprofen suspension

| Test method | Descriptive and discriminative | |
|---|---|---|
| | evaluation, “sip and spit” method, | |
| | blinded and randomised samples | |
| Test sample | Purified dehydroabietic acid, purity > 98% | |
| | ibuprofen suspension: suspension with orange | |
| | taste, no sweeteners | |
| Panelists | 5 experienced assessors | |
| Preparation | (A) | Dehydroabietic acid, dissolved in |
| of the samples | | ethanol |
| | (B) | Ibuprofen suspension, dissolved in |
| | | water, final concentration of |
| | | ibuprofen was 5 mg/ml |

| | |
|---|---|
| | Sample: (A) + (B), final 25 μM |
| | dehydroabietic acid, 0.5% ethanol |
| | Comparison sample: ibuprofen suspension, |
| | dissolved in water with 0.5% ethanol |
| Evaluation of the sample | less bitter |
| Ibuprofen suspension, | more sweet |
| dissolved in water plus | intensification of the orange aroma |
| 25 μM dehydroabietic | more pleasant taste |
| acid | |

### Example 6

Taste Modulation of Ibuprofen in a Maltitol Formulation

The sensory evaluation of the samples was performed by a team of five experienced assessors. Details may be taken from the following Table 6:

TABLE 6
Tasting of a maltitol/ibuprofen solution

| Test method | Descriptive and discriminative | |
|---|---|---|
| | evaluation, “sip and spit” | |
| | method, blinded and randomised samples | |
| Test sample | Purified dehydroabietic acid, purity > 98% | |
| | Suspension of ibuprofen, dissolved | |
| | in maltitol syrup | |
| Panelists | 5 experienced assessors | |
| Preparation | (A) | Dehydroabietic acid, dissolved in |
| of the samples | | ethanol |
| | (B) | Ibuprofen/maltitol syrup, dissolved in |
| | | water, final concentration of |
| | | Paracetamol was 5 mg/ml |

| | |
|---|---|
| | Sample: (A) + (B), final 25 μM |
| | dehydroabietic acid, 0.5% ethanol |
| | Comparison sample: ibuprofen syrup in |
| | water with 0.5% ethanol |
| Evaluation of the sample | less bitter |
| Ibuprofen/maltitol syrup | less unpleasant aftertaste |
| in water plus 25 μM | reduced mouth dryness |
| dehydroabietic acid | slightly sweet |
| | alleviation of the burning sensation |
| | on the tongue/in the throat |

### FORMULATION EXAMPLES

Effervescent Tablet

500 mg paracetamol

5 mg dehydroabietic acid

200 mg ascorbic acid

50 mg sodium cyclamate

8 mg sodium saccharinate

327 mg sorbitol

30 mg fruit flavour

20 mg propylene glycol

1.160 mg citric acid

1.000 mg sodium carbonate

Weight of the effervescent tablet 3.3 g, which corresponds to a content in active ingredients of 15%.

Retard Tablet

500 mg acetylsalicylic acid

1 mg dehydroabietic acid

100 mg maize starch or pregelatinised maize starch

Weight of the tablet 600 mg, which corresponds to a content in active ingredients of 83%.

Gelatin Capsule or Alginate Capsule

Ibuprofen and dehydroabietic acid are granu led along with Colestipol and filled into a gelatin capsule (333 mg weight with 200 mg active ingredient, which corresponds to a content in active ingredients of 60%).

Tablet

256.25 kg ibuprofen sodium dihyd rate, 0.2 kg dehydroabietic acid, 25 kg povidone K25 and 46.75 kg sodium hydrogencarbonate are compressed into tablets of 331 mg each. The content in active ingredients is ca. 78%.

1. A preparation, comprising
(a) at least one NSAID active ingredient, and
(b) dehydroabietic acid or an extract containing dehydroabietic as a pharmaceutical.

2. The preparation of claim 1, in an amount effective
for the treatment and/or the prophylaxis of pain conditions, inflammatory conditions and rheumatic diseases.

3. The preparation of claim 1, wherein the NSAID active ingredient is selected from the group consisting of acetylsalicylic acid derivatives, arylpropionic acid derivatives, arylacetic acid derivatives, indoleacetic acid derivatives, anthranilic acid derivatives, oxicams and mixtures thereof.

4. The preparation of claim 3, wherein the NSAID active ingredient is selected from the group consisting of acetylsalicylic acid (Aspirin), ibuprofen, flurbiprofen, naproxen, ketoprofen, tiaprofenic acid, diclofenac, indometacin, flufenamic acid, mefenamic add, piroxicam, tenoxicam, meloxicam, para-acetylaminophenol (paracetamol) and mixtures thereof.

5. The preparation of claim 1, additionally comprising at least one sweetener (component (c)).

6. The preparation of claim 5, wherein the sweetener, is selected from the group consisting of saccharose, trehalose, lactose, maltose, melizitose, raffinose, palatinose, lactulose, D-fructose, D-glucose, D-galactose, L-rhamnose, D-sorbose, D-mannose, D-tagatose, D-arabinose, L-arabinose, D-ribose, D-glyceraldehyde or maltodextrin. Plant-based preparations containing these substances are also suitable, for example, on the basis of sugarbeet (Beta vulgaris ssp., sugar fractions, sugar syrup, molasses), sugar cane (Saccharum officinarum ssp., molasses, sugar cane syrup), maple syrup (Acer ssp.) or agave (agave syrup), fruit concentrates (e.g., on the basis of apples or pears); erythritol, threitol, arabitol, ribotol, xylitol, sorbitol, mannitol, dulcitol, lactitol, miraculin, monellin, thaumatin, curculin, brazzein, magap, sodium cyclamate, acesulfame-K, neohesperidin dihydrochalcone, naringin-dihydrochalcone, saccharine sodium salt, aspartame, superaspartame, neotame, alitame, sucralose, stevioside, rebaudioside, lugduname, carrelame, sucrononate, sucrooctate, monatin, phenylodulcin, glycine, D-leucine, D-threonine, D-asparagine, D-phenylalanine, D-tryptophan, L-proline, hernandulcin, phyllodulcin, dihydrochalcon glycoside, glycerrhetinic acid and its derivatives and salts, extracts of licorice (Glycyrrhiza ssp.), Lippia dulcis extracts, Momordica ssp. extracts, mogrosides, extracts of the plants of the genus Rubus, and mixtures thereof.

7. The preparation of claim 1, additionally comprising at least one carrier (component (d)).

8. The preparation of claim 7, comprising either a solid carrier selected from the group consisting of dextrins, celluloses, microcrystalline celluloses, starches, modified starches, Xanthan Gum and mixtures thereof, or a liquid carrier selected from the rot consisting of water, ethanol, glycerol, ethylene glycol and mixtures thereof.

9. The preparation of claim 1, in the form of a solid or liquid formulation.

10. The preparation of claim 9, in the form of tablets, lozenges, chewing gums or capsules.

11. The preparation of claim 9, in the form of a syrup or a spray.

12. The preparation of claim 1, comprising
(a) about 0.01 to about 90% by weight NSAID active ingredient,
(b) about 0.001 to about 5% by weight dehydroabietic acid or a corresponding extract,
(c) 0 to about 15% by weight sweetener, and
(d) 0 to about 95% by weight carrier,
with the proviso that all quantities, optionally with further additives and adjuvants, add up to 100% by weight.

13. A process for masking the unpleasant taste of an NSAID active ingredient and a formulation containing said active ingredient, comprising form sating the active ingredient along with dehydroabietic acid or an extract containing dehydroabietic acid, and then administering the resulting formulation orally.

14. A process for alleviating and/or reducing the burning sensation caused by an NSAID active ingredient and a formulation containing said active ingredient in the throat and on the tongue as well as for increasing mouth moisture, comprising formulating the active ingredient along with dehydroabietic acid or an extract containing dehydroabietic acid, and then administering the resulting formulation orally.

15. A method for
masking the bitter and/or metallic taste of an NSAID active ingredient and, optionally, further formulation components,
intensifying the aromas and the sweetness of a formulation containing the NSAID active ingredient
and improving of the mucous membrane compatibility during oral uptake of the formulation containing the NSAID active ingredient comprising with administering the preparation on of claim 1.
